(19) United States
(12) Patent Application Publication
(10) Pub. No.: US 2018/0201579 A1
(43) Pub. Date: Jul. 19, 2018

(54) BISMALEIMIDE AND PRODUCTION METHOD THEREOF

(71) Applicant: UNITIKA LTD., Amagasaki-shi (JP)

(72) Inventors: Yuki YAMADA, Uji-shi (JP); Tatsuya MORIKITA, Uji-shi (JP); Takeshi YOSHIDA, Uji-shi (JP); Akira SHIGETA, Uji-shi (JP); Yoshiaki ECHIGO, Uji-shi (JP)

(73) Assignee: UNITIKA LTD., Amagasaki-shi (JP)

(21) Appl. No.: 15/872,463

(22) Filed: Jan. 16, 2018

(30) Foreign Application Priority Data
Jan. 17, 2017 (JP) 2017-005954

(51) Int. Cl. C07D 207/452; C07C 267/00; B01D 11/04

(52) U.S. Cl. CPC C07D 207/452; B01D 11/0492; C07C 267/00

(57) ABSTRACT

An object of the present invention is to provide a bismaleimide using an aliphatic diamine, in which acid components remaining as an impurity at a very small amount in the bismaleimide have been effectively removed, that is, a bismaleimide using an aliphatic diamine, an acid value of which has sufficiently been reduced, and a method for producing it. There are provided a bismaleimide using an aliphatic diamine as a diamine component, characterized in that an acid value thereof is 2 mg-KOH/g or less, and a method for producing the bismaleimide, comprising adding carbodiimide to a solution comprising a crude bismaleimide having an acid value of more than 2 mg-KOH/g, and reacting acid components in the crude bismaleimide with carbodiimide.

### ART FIELD RELATED

The present invention relates to a bismaleimide (hereinafter, abbreviated as “BMI” in some cases) useful as a laminate material, a seal material, an electrical insulating material, an electrically conductive paste, an adhesive, a pressure-sensitive adhesive, a structural material and the like, and a method for producing the same.

### PRIOR ART

BMI is produced, for example, by reacting diamine with maleic anhydride under an acid catalyst in a solvent to form maleamic acid (hereinafter, abbreviated as “MAA” in some cases), maleimidizing (ring-closing by dehydration) this with an acid catalyst or the like to obtain a crude BMI solution, and purifying this, and is widely utilized as a laminate material, a seal material, an electrical insulating material, an electrically conductive paste, an adhesive, a pressure-sensitive adhesive, a structural material and the like. In the produced BMI, acid components such as MAA, fumaric acid, and a Michael adduct (compound formed by reacting a compound which has been formed-by a Michael addition reaction of amine to MAA, further with maleic anhydride) remain as an impurity at a very small amount, and these acid components become the cause for generating corrosion resulting from BMI in a semiconductor device, in some cases, when used as an adhesive or a pressure-sensitive adhesive for semiconductors. Additionally, hygroscopicity of BMI due to the acid components is increased, and this causes deterioration of electric properties, in some cases. Then, methods of removing acid components remaining at a very small amount in a step of purifying BMI, that is, methods of reducing an acid value of BMI have variously been proposed. In addition, Non-Patent Document 1 describes that the acid components as described above are contained in a crude maleimide solution which is obtained by reacting amine with maleic anhydride to form maleamic acid, and thereafter, maleimidizing-maleamic acid with an acid catalyst or the like.

For example, concerning BMI using an aromatic diamine as a diamine, a method of obtaining BMI having an acid value of 2 mg-KOH/g or less by purifying a crude BMI solution before purification by a method such as crystallization, reprecipitation, and water washing is disclosed in Patent Documents 1 to 6.

On the other hand, concerning BMI using an aliphatic diamine, Patent Documents 7 to 12 disclose various BMIs using the aliphatic diamines such as dimer diamine described later. Additionally, Patent Documents 11 and 12 disclose BMI using an imide-extended aliphatic diamine. Since BMI using the aliphatic diamines is excellent in a toughness and dielectric properties as compared with BMI using the aromatic diamines, use thereof in the semiconductor field is expected. These BMIs using the aliphatic diamines can be obtained by using, as a dehydration catalyst of MAA, formic acid, methanesulfonic acid, a cationic ion exchange resin acid or a salt thereof. Alternatively, as a dehydration catalyst of MAA, a mixture consisting of dicyclohexylcarbodiimide and 1-hydroxybenzotriazole can also be used. These BMIs using the aliphatic diamines are also commercially available under a product name of BMI-689, BMI-1500, BMI-1700, BMI-3000 or the like, from Designer Molecules Inc. (hereinafter, abbreviated as “DMI” in some cases).

### PRIOR ART DOCUMENTS

### Patent Documents

[Patent Document 1] JP-A No. 01-238568

[Patent Document 2] JP-A No. 03-145462

[Patent Document 3] JP-A No. 06-345730

[Patent Document 4] JP-A No. 07-002768

[Patent Document 5] JP-A No. 07-118230

[Patent Document 6] JP-A No. 08-119939

[Patent Document 7] US Statutory Invention Registration No. H424

[Patent Document 8] US Patent No. 6281314

[Patent Document 9] US Patent No. 5973166

[Patent Document 10] JP-A No. 10-505599

[Patent Document 11] US 2008/0262191

[Patent Document 12] JP-A No. 2012-117070

### Non-Patent Documents

[Non-Patent Document 1] Bulletin of the Chemical Society of Japan, 1996, (4), pp. 375-384

### DISCLOSURE OF INVENTION

### Technical Problems to be Solved

However, since acid components such as MAA in which maleimide rings have not yet formed remain at a very small amount in BMI using the aliphatic diamine which has been obtained by the known method, an acid value of the BMI considerably exceeded 2 mg-KOH/g. In this BMI, an acid value cannot be reduced to the level of 2 mg-KOH/g or less at which use in the field of semiconductors or the like is possible, even when the known purification method such as the above-mentioned crystallization, reprecipitation, and solvent extraction by water washing is fully used, and BMI using the aliphatic diamine, which has an acid value of 2 mg-KOH/g or less, has not hitherto been known. Additionally, also regarding the above-mentioned commercially available product of “DMI”, an acid value exceeded 2 mg-KOH/g.

Then, an object of the present invention is to provide BMI using the aliphatic diamines, in which acid components remaining as an impurity at a very small amount in BMI have been effectively removed, that is, BMI using the aliphatic diamines, an acid value of which has sufficiently been reduced, and a method for producing the same.

### Means to Solve the Problems

It was found out that by reacting acid components remaining at a very small amount in a crude BMI with a specific compound, an acid value of the crude BMI is remarkably reduced, and BMI having a low acid value at the level which has not previously been known is obtained, resulting in completion of the present invention.

The present invention has the following gist.

<1>A BMI using an aliphatic diamine as a diamine component, characterized in that an acid value thereof is 2 mg-KOH/g or less.

<2>A method for producing the BMI, comprising: adding a carbodiimide (hereinafter, abbreviated as “CDI” in some cases) to a solution comprising a crude BMI having an acid value of more than 2 mg-KOH/g; and
reacting acid components in the crude BMI with CDI to give BMI having an acid value of 2 mg-KOH/g or less.

<3>A method for producing the BMI as defined in claim 1, comprising: adding CDI to a solution comprising a crude BMI having an acid value of more than 2 mg-KOH/g;
reacting acid components in the crude BMI with CDI; and thereafter, removing a carbodiimide urea derivative (hereinafter, abbreviated as “CDI-U” in some cases).

<4>The method for producing the BMI, wherein CDI-U is soluble in methanol.

<5>The method for producing the BMI, wherein the method of removing CDI-U is a solvent extraction method.

### Effects of the Invention

Since BMI of the present invention has a remarkably low acid value, the BMI has an excellent corrosion resistance, and has a low hygroscopicity. Accordingly, the BMI can be suitably used as a component of an adhesive, a pressure-sensitive adhesive, a sealant and the like, which are applied to semiconductors and the like.

### BEST MODE FOR CARRYING OUT THE INVENTION

The present invention will be explained in detail below.

In BMI of the present invention, as a diamine component, an aliphatic diamine is used. Herein, BMI using the aliphatic diamines refers to BMI in which a diamine component constituting BMI comprises an aliphatic diamine.

Specific examples of the aliphatic diamine include, for example, 1,4-diaminobutane, 1,5-diaminopentane, 1,6-diaminohexane, 2-methyl-1,5-diaminopentane, 1,7-diaminoheptane, 1,8-diaminooctane, 1,9-diaminononane, 1,10-diaminodecane, 1,11-diaminoundecane, 1,12-diaminododecane, 1,3-bis(aminomethyl)cyclohexane, 1,4-bis(aminomethyl)cyclohexane, 4,4′-methylenebiscyclohexylamine, dimer diamine (hereinafter, abbreviated as DDA in some cases) and the like. These may be used alone, or two or more may be used by combining them. Among them, DDA is preferable. DDA is an aliphatic diamine which is derived from a dimer acid having 24 to 48 carbon atoms. DDA is obtained, for example, by polymerizing an unsaturated fatty acid such as oleic acid and linoleic acid to form a dimer acid, and reducing and aminating this (reductive amination). As DDA, commercially available products such as “Priamine 1074, the same 1075” (product names made by Croda Japan KK), and “Versamine 551, the same 552” (product names made by Cognis Japan Ltd.) can be used.

As the aliphatic diamine, the “imide-extended diamine” described in Patent Documents 11 and 12 can also be preferably used. Herein, the imide-extended diamine refers to “polyimide or oligoimide having an amino group on both end groups” (hereinafter, abbreviated as “ATPI” in some cases) which is obtained by reacting a tetracarboxylic dianhydride with an excessive amount of the aliphatic diamide to perform dehydration and ring-closure. Examples of the tetracarboxylic dianhydride include pyromellitic dianhydride (PMDA), 3,3′,4,4′-biphenyltetracarboxylic dianhydride, 2,3,3′,4′-biphenyltetracarboxylic dianhydride, 3,3′,4,4′-benzophenonetetracarboxylic dianhydride (BTDA), 4,4′-oxydiphthalic anhydride (ODPA), 2,2-bis(3,4-dicarboxyphenyl)propane dianhydride (BDCP), 3,3′,4,4′-diphenylsulfonetetracarboxylic dianhydride and the like. These may be used alone, or two or more may be used by combining them. Among them, PMDA, ODPA, and BTDA are preferable. Additionally, as the aliphatic diamine, the above-mentioned aliphatic diamine can be used, and DDA is preferable.

The aliphatic diamine can also be used by mixing with an aromatic diamine (including a heterocyclic diamine). Specific examples of the aromatic diamine include, for example, 4,4′-diaminodiphenyl ether,
2′-methoxy-4,4′-diaminobenzanilide, 1,4-bis(4-aminophenoxy)benzene,
1,3-bis(4-aminophenoxy)benzene,
2,2′-bis[4-(4-aminophenoxy)phenyl]propane,
2,2′-dimethyl-4,4′-diaminobiphenyl, 3,3′-dihydroxy-4,4′-diaminobiphenyl,
4,4′-diaminobenzanilide, bisanilinefluorene,
2,2-bis-[4-(3-aminophenoxy)phenyl]propane,
bis[4-(4-aminophenoxy)phenyl]sulfone,
bis[4-(3-aminophenoxy)phenyl]sulfone, bis[4-(4-aminophenoxy)]biphenyl,
bis[4-(3-aminophenoxy)biphenyl, bis[1-(4-aminophenoxy)]biphenyl,
bis[1-(3-aminophenoxy)]biphenyl, bis[4-(4-aminophenoxy)phenyl]methane,
bis[4-(3-aminophenoxy)phenyl]methane, bis[4-(4-aminophenoxy)phenyl] ether, bis[4-(3-aminophenoxy)phenyl] ether,
bis[4-(4-aminophenoxy)]benzophenone,
bis[4-(3-aminophenoxy)]benzophenone,
bis[4,4′-(4-aminophenoxy)]benzanilide,
bis[4,4′-(3-aminophenoxy)]benzanilide,
9,9-bis[4-(4-aminophenoxy)phenyl]fluorene,
9,9-bis[4-(3-aminophenoxy)phenyl]fluorene,
2,2-bis-[4-(4-aminophenoxy)phenyl]hexafluoropropane,
2,2-bis-[4-(3-aminophenoxy)phenyl]hexafluoropropane,
4,4′-methylenedi-o-toluidine, 4,4′-methylenedi-2,6-xylidine,
4,4′-methylene-2,6-diethylaniline, 4,4′-diaminodiphenylpropane,
3,3′-diaminodiphenylpropane, 4,4′-diaminodiphenylethane,
3,3′-diaminodiphenylethane, 4,4′-diaminodiphenylmethane,
3,3′-diaminodiphenylmethane, 4,4′-diaminodiphenyl sulfide,
3,3′-diaminodiphenyl sulfide, 4,4′-diaminodiphenylsulfone,
3,3′-diaminodiphenylsulfone, 3, 3-diaminodiphenyl ether,
3,4′-diaminodiphenyl ether, benzidine, 3,3′-diaminobiphenyl,
3,3′-dimethyl-4,4′-diaminobiphenyl, 3,3′-dimethoxybenzidine,
4,4″-diamino-p-terphenyl, 3,3″-diamino-p-terphenyl, m-phenylenediamine,
p-phenylenediamine, 2,6-diaminopyridine, 1,4-bis(4-aminophenoxy)benzene,
1,3-bis(4-aminophenoxy)benzene,
4,4′-[1,4-phenylenebis(1-methylethylidene)]bisaniline,
4,4′-[1,3-phenylenebis(1-methylethylidene)]bisaniline,
bis(p-aminocyclohexyl)methane, bis(p-ß-amino-t-butylphenyl) ether,
bis(p-ß-methyl-δ-aminopentyl)benzene,
p-bis(2-methyl-4-aminopentyl)benzene,
p-bis(1,1-dimethyl-5-aminopentyl)benzene, 1,5-diaminonaphthalene,
2,6-diaminonaphthalene, 2,4-bis(ß-amino-t-butyl)toluene,
2, 4-diaminotoluene, m-xylene-2,5-diamine, p-xylene-2,5-diamine,
m-xylilenediamine, p-xylilenediamine, 2,6-diaminopyridine,
2,5-diaminopyridine, 2,5-diamino-1,3,4-oxadiazole and the like. These may be used alone, or two or more may be used by combining them.

A used amount of these aromatic diamines is preferably 20 mol % or less, and more preferably 10 mol % or less, based on a total amount of diamines used.

BMI of the present invention has an acid value of 2 mg-KOH/g or less. This acid value is a value which was measured by a neutralization titration method, based on the provision of JIS K0070 (1992). An acid value of BMI is preferably 1.5 mg-KOH/g or less, and more preferably 1.0 mg-KOH/g or less. By doing this, a good corrosion resistance and a low hygroscopicity of BMI can be secured. Herein, the BMI having an acid value of 2 mg-KOH/g or less means that an acid value as a composition comprising BMI and the above-mentioned acid components remaining in BMI at a very small amount (MAA, fumaric acid, Michael adduct etc.) is 2 mg-KOH/g or less.

The BMI of the present invention can be obtained, for example, by reacting the aliphatic diamine with maleic anhydride in the presence of an acid catalyst in a solvent to obtain a crude BMI solution, and reacting the acid components remaining at a very small amount with CDI at a next purification step, thereby, reducing an acid value. The crude BMI solution may be, for example, a solution of unpurified BMI, which is obtained by reacting the aliphatic diamine with maleic anhydride in the known method described later to obtain MAA, and thereafter, performing dehydration and ring-closure (maleimidization), or a solution of BMI which is obtained by purifying the relevant unpurified BMI solution by the known method such as crystallization, reprecipitation, and water washing. As a catalyst for dehydration and ring-closure, in addition to the above-mentioned acid catalyst, a mixture comprising of acetic anhydride, dicyclohexylcarbodiimide and 1-hydroxybenzotriazol or the like can also be used.

In order to obtain the crude BMI solution, the known methods can be used. That is, for example, the crude BMI solution can be obtained by reacting the aliphatic diamine with an approximately equal equivalent of maleic anhydride at a temperature of 0° C. to 50° C. in a solvent to obtain MAA, and thereafter, dehydrating and-ring-closing (maleimidizing) this at a temperature of 50° C. to 200° C. under an acid catalyst. The solvent used is not limited, and a hydrocarbon-based solvent such as toluene, xylene (o-xylene, m-xylene, p-xylene), ethylbenzene, and mesitylene, an amide-based solvent such as N,N-dimethylformamide, N,N-dimethylacetamide, and N-methyl-2-pyrrolidone (NMP), a mixed solvent of the hydrocarbon-based solvent and the amide-based solvent, and the like are preferable.

Additionally, the acid catalyst used is not limited, and sulfuric acid, formic acid, methanesulfonic acid, benzenesulfonic acid, orthophosphoric acid, metaphosphoric acid, pyrophosphoric acid, phosphorous acid, hypophosphoric acid, maleic acid, a cationic ion exchange resin and the like can be used. Triethylamine salts of these acids can also be used. When maleic acid is used as the acid catalyst, maleic acid which is generated by hydrolysis of maleic anhydride used as a raw material of BMI synthesis during a reaction is also included. Accordingly, when a largely excessive maleic anhydride is used as a raw material, it is not necessary to use an acid catalyst separately. When dehydration and ring-closure are performed, it is preferable to remove water generated by maleimidization to the outside of a reaction system by azeotropy or the like. In addition, as a method of obtaining the crude BMI solution when ATPI is used as a diamine, a method described in Patent Documents 11 and 12 can be referenced. Details of this method are shown in Reference Example described later.

In a method for producing the BMI of the present invention, by reacting the acid components in the crude BMI remaining as an impurity at a very small amount, from the crude BMI solution obtained as described above, with CDI, an acid value of the crude BMI can remarkably be reduced. When acid components in the crude BMI and CDI are reacted, as main products, a maleimide in which acid components are dehydrated and ring-closed, and CDI-U are formed. BMI containing this CDI-U can be used as it is, but it is preferable to use BMI from which CDI-U has been removed. In addition, O-acylisourea or N-acylurea which is an acylurea derivative of acid components remaining in the crude BMI may have been generated. Also by generation of the acylurea derivative, an acid value of BMI is reduced. Concerning generation of CDI-U and the acylurea derivative by CDI, Tetrahedron 63 (28) 6508˜6511 (2007) and the like can be referenced.

As the crude BMI, BMI which is commercially available as an industrial chemical product from “DMI” or the like, BMI which is commercially available as a reagent and the like can also be used.

As CDI, monocarbodiimide, polycarbodiimide, cyclic carbodiimide and the like can be used. Specific examples of the monocarbodiimide include, for example, bis(2,6-diisopropylphenyl)carbodiimide,
diphenylcarbodiimide, di-ß-naphthylcarbodiimide,
N,N′-diisopropylcarbodiimide (DIC),
1-ethyl-3-(3-dimethylaminopropyl)carbodiimide (EDC),
dimethylcarbodiimide, diisobutylcarbodiimide, dioctylcarbodiimide,
t-butylisopropylcarbodiimide, di-t-butylcarbodiimide,
N,N′-dicyclohexylcarbodiimide (DCC) and the like. Specific examples of the polycarbodiimide (number average molecular weight: 300 to 20000) include, for example, poly(1,6-hexamethylenecarbodiimide),
poly(4,4′-methylenebiscyclohexylcarbodiimide),
poly(1,3-cyclohexylenecarbodiimide), poly(l,4-cyclohexylenecarbodiimide),
poly(4,4′-dicyclohexylmethanecarb odiimide),
poly(4,4′-diphenylmethanecarbodiimide),
poly(3,3′-dimethyl-4,4′-diphenylmethanecarbodiimide),
poly(naphthylenecarbodiimide), poly(p-phenylenecarbodiimide),
poly(m-phenylenecarbodiimide), poly(tolylcarbodiimide),
poly(methyl-diisopropylphenylenecarbodiimide),
poly(1,3,5-triisopropylbenzene)carbodiimide,
copoly(1,3,5-triisopropylbenzene/1,5-diisopropylbenzenecarbodiimide), poly(triethylphenylenecarbodiimide),
poly(triisopropylphenylenecarbodiimide), poly(diisopropylcarbodiimide) and the like. These may be used alone, or two or more may be used by combining them. Among carbodiimides, monocarbodiimides generating a carbodiimide derivative such as CDI-U generated as a byproduct after the reaction which can be easily removed are preferable. Among monocarbodiimides, DIC and EDC are more preferable, and DIC is particularly preferable. In addition, as the polycarbodiimide, commercially available products such as “CARBODILITE” (product name made by Nisshinbo Chemical Inc.), and “Stabaxol” (product name made by Rhein Chemie) can be used. Additionally, as the cyclic carbodiimide, “TCC” (product name made by TEIJIN LIMITED.) can be used.

In order to react the acid components in the crude BMI remaining at a very small amount with CDI, CDI is added at 0.5 to 10% by mass based on the mass of BMI, and they are reacted in a solvent. A reaction temperature at that time is preferably 50 to 150° C., and more preferably 70 to 120° C. The BMI concentration at the reaction is preferably 20 to 70% by mass. A reaction solvent is preferably a hydrocarbon-based solvent such as toluene, xylene, heptane, and octane. By reacting the acid components in the crude BMI with CDI in this way, BMI having 2 mg-KOH/g or less can be obtained. In addition, since when CDI is reacted with the acid components in BMI, CDI-U is generated as a byproduct as described above, it can be removed from a BMI solution by methods such as reprecipitation, filtration, and solvent extraction.

For removing CDI-U, it is preferable to use a solvent extraction method which is simple in operation. As an extracting agent used at the time of solvent extraction, water, methyl alcohol, ethyl alcohol, acetone and the like can be used alone, or can be used by mixing them, and methanol is preferable. In addition, since CDI-U derived from the above-mentioned DIC or EDC is well soluble in these extracting agents, when CDI-U is removed by the solvent extraction method, it is preferable to use DIC or EDC.

When after a reaction of the acid components in the crude BMI with CDI, CDI-U is precipitated in a reaction solution, CDI-U can be removed by a filtration method.

CDI-U which has been removed can be regenerated into CDI by performing the known method, for example, a dehydration reaction using a dehydration catalyst, and regenerated CDI can be repeatedly used.

In addition, a method of removing the acid components from the crude BMI using CDI in the production method of the present invention can also be applied when BMI is BMI using an aromatic diamine as a diamine component. That is, by adding CDI to a solution comprising the crude BMI having an acid value of more than 2 mg-KOH/g, and reacting the acid components in the crude BMI using the aromatic diamine as a diamine component with carbodiimide, and thereafter, removing CDI-U, a BMI solution having an acid value of 2 mg-KOH/g or less can easily be obtained. As the aromatic diamine used herein, the above mentioned aromatic diamine can be used.

### EXAMPLES

Examples of the present invention will be explained in detail below, but the present invention is not limited to only these Examples.

In the following Reference Examples, Preparation Examples of crude BMI solutions used in Examples and Comparative Examples will be shown.

### Reference Example 1

In accordance with the description of Example 1 of Patent Document 12, a crude BMI solution was prepared as follows. That is, 250 ml of toluene, 0.35 mole of triethylamine, and 0.36 mole of methanesulfonic acid were added to a reactor vessel, and stirred. Then, 0.11 mole of Versamine 552 (DDA made by Cognis Japan Ltd., molecular weight is 520) and 0.05 mole of PMDA were added while stirring. A Dean-Stark trap and a condenser were attached to the reactor vessel, a mixture was refluxed for 2 hours, and water generated by imidization was removed to the outside of a system, thereby, ATPI was obtained. The reaction mixture was cooled to room temperature, 0.13 mole of maleic anhydride was added to the reactor vessel, and subsequently, 0.05 mole of methanesulfonic acid was added. The mixture was further refluxed for 12 hours, and water generated by maleimidization was removed to the outside of a reaction system. After cooled to room temperature, 100 ml of toluene was further added to generate the precipitate. The precipitate was removed by filtration, thereby, a toluene solution of BMI was obtained. This solution was added to a large amount of methanol, BMI was reprecipitated, and the precipitate was dissolved in toluene, thereby, a crude BMI (C-1) solution (L-1) having the BMI concentration of 50% by mass was obtained. An acid value of C-1 was measured based on the provision of JIS K0070 (1992), and as a result, an acid value was found to be 9.52 mg-KOH/g.

### Reference Example 2

In accordance with the description of Example 2 of Patent Document 12, a crude BMI solution was prepared as follows. That is, 250 ml of toluene, 0.38 mole of triethylamine, and 0.39 mole of methanesulfonic acid were added to a reactor vessel, mixed. Then, 0.11 mole of Versamine 552 and 0.05 mole of BTDA were added while stirring. A Dean-Stark trap and a condenser were attached to the reactor vessel, and the mixture was refluxed for 15 hours to remove water generated by imidization to the outside of a system. The reaction mixture was cooled to room temperature, 0.23 mole of maleic anhydride was added to the reactor vessel, the mixture was refluxed for 12 hours to remove water generated by maleimidization to the outside of a reaction system, thereby, a toluene solution of BMI was obtained. After cooled to room temperature, 100 ml of toluene was further added to generate the precipitate. The precipitate was removed by filtration, thereby, a toluene solution of BMI was obtained. This solution was added to a large amount of methanol to reprecipitate BMI, and the precipitate was collected, mixed with toluene, thereby, a crude BMI (C-2) solution (L-2) having the BMI concentration of 50% by mass was obtained. An acid value of C-2 was found to be 8.61 mg-KOH/g.

### Reference Example 3

According to the same manner as that of Reference Example 2 except that “0.11 mole of Versamine 552” was changed to “a mixture of 0.105 mole of Versamine and 0.05 mole of 2,2′-bis[4-(4-aminophenoxy)phenyl]propane”, a crude BMI (C-3) solution (L-3) having the BMI concentration of 50% by mass was obtained. An acid value of C-3 was found to be 9.17 mg-KOH/g.

### Reference Example 4

In accordance with the description of Example 1 of Patent Document 10, a crude BMI solution was prepared as follows. That is, a solution obtained by dissolving 0.058 mole of Versamine 552 in 90 ml of tetrahydrofuran (THF) was added slowly into a solution obtained by dissolving 0.127 mole of maleic anhydride in 60 ml of THF. After 1 hour from addition, 125 mL of acetic anhydride was added, and this reaction mixture was stirred for 24 hours. This reaction mixture was refluxed, and retained at the refluxing temperature for 3 hours. To this reaction mixture was added 0.1 g of benzoquinone, and thereafter, a solvent was removed under vacuum. To the resulting residue were added 75mL of THF and 1-hydroxybenzotriazole (HOBt), and the materials were dissolved at room temperature, and stirred for 24 hours. Thereafter, the solvent was removed at 30° C., and the residue was extracted with 500 mL of pentane two times. Since when these pentane portions were combined, and it was cooled in a dry ice/isopropyl alcohol bath, a white solid was crystallized, this was filtered under cooling, and concentrated, thereby, a crude BMI (C-4) solution (L-4) having the BMI concentration of 50% by mass was obtained. An acid value of C-4 was found to be 9.83 mg-KOH/g. A solution of the crude BMI was obtained.

### Reference Example 5

In accordance with the description of Example 3 of Patent Document 10, a crude BMI solution was prepared as follows. That is, a solution obtained by dissolving 0.096 mole of Versamine 552 in 60 mL of THF was added slowly to a solution obtained by dissolving 0.206 mole of maleic anhydride in 300 mL of THF. After completion of addition, this reaction mixture was stirred for 1 hour, and then, HOBt was dissolved therein. This stirred reaction mixture was cooled in an ice bath, and thereafter, 0.238 mole of DCC was added by small and small. After completion of this addition, the reaction mixture was further stirred in an ice bath for 1 hour. Then, the ice bath was removed, and the stirred reaction mixture was warmed to room temperature overnight. This reaction mixture was filtered, and the resulting solid was washed with THF. All of these THF portions were combined, 0.2 g of methoxyphenol was added thereto, and thereafter, THF was removed at 30° C. This residue was extracted with hexane, and the hexane was removed. Then, this was extracted with pentane again, thereby, a crude BMI (C-5) solution (L-5) having the BMI concentration of 50% by mass was obtained. An acid value of C-5 was found to be 6.68 mg-KOH/g.

### Example 1

DIC (1g) was added to 100 g of a crude BMI solution (L-1), and the materials were reacted at 100° C. for 5 hours. When after cooling, an acid value of BMI in the reaction solution was measured, it was found to be 0.84 mg-KOH/g. A liquid obtained by adding toluene to this reaction liquid was added to a large amount of methanol under stirring to reprecipitate BMI and to dissolve a urea derivative of DIC which was generated as a byproduct in methanol for removing it, and thereafter, the precipitate was mixed with toluene, thereby, a purified BMI (P-1) solution having the BMI concentration of 50% by mass was obtained. The result of measurement of an acid value of P-1 is shown in Table 1. In addition, it was confirmed by measuring NMR of a methanol phase that a urea derivative of DIC which had been generated as a byproduct was transferred to the methanol phase by solvent extraction with methanol.

### Example 2

According to the same manner as that of Example 1 except that an addition amount of DIC was 1.5 g, a purified BMI (P-2) solution was obtained. The result of measurement of an acid value of P-2 is shown in Table 1.

### Example 3

According to the same manner as that of Example 1 except that an addition amount of DIC was 0.8 g, a purified BMI (P-3) solution was obtained. The result of measurement of an acid value of P-3 is shown in Table 1.

### Example 4

DCC (4g) was added to 100 g of a crude BMI solution (L-1), and the materials were reacted at 100° C. for 5 hours. After cooling, toluene was added, a urea derivative of DCC which was generated as a byproduct was removed by filtration using a membrane filter to obtain a purified BMI (P-4) solution. The result of measurement of an acid value of P-4 is shown in Table 1.

### Example 5

According to the same manner as that of Example 1 except that a crude BMI solution was L-2, a purified BMI (P-5) solution was obtained. The result of measurement of an acid value of P-5 is shown in Table 1.

### Example 6

According to the same manner as that of Example 4 except that a crude BMI solution was L-2, a purified BMI (P-6) solution was obtained. The result of measurement of an acid value of P-6 is shown in Table 1.

### Example 7

According to the same manner as that of Example 1 except that a crude BMI solution was L-3, a purified BMI (P-7) solution was obtained. The result of measurement of an acid value of P-7 is shown in Table 1.

### Example 8

As a crude BMI solution, a toluene solution (concentration: 50% by mass) of BMI-689 which is commercially available from “DMI” was prepared. This BMI is BMI using DDA as a diamine component, and had an acid value of 3.12 mg-KOH/g. Using this crude BMI solution, a reaction with DIC was performed under the same condition as that of Example 1, and after cooling, an acid value of BMI in the reaction solution was measured, and it was found to be 0.27 mg-KOH/g. Then, a urea derivative of DIC was removed from this solution by solvent extraction with methanol according to the same manner as that of Example 1, to obtain a purified BMI (P-8) solution. The result of measurement of an acid value of P-8 is shown in Table 1.

### Example 9

As a crude BMI solution, a toluene solution (concentration: 50% b_{y} mass) of BMI-1500 which is commercially available from “DMI” was prepared. This BMI is BMI using DDA which was imide-extended with ODPA, as a diamine component, and had an acid value of 6.44 mg-KOH/g. According to the same manner as that of Example 1, this BMI was used to obtain a purified BMI (P-9) solution. The result of measurement of an acid value of P-9 is shown in Table 1.

### Example 10

As a crude BMI solution, a toluene solution (concentration: 50% by mass) of BMI-1700 which is commercially available from “DMI” was prepared. This BMI is BMI using DDA which was imide-extended with BDCP, as a diamine component, and had an acid value of 5.01 mg-KOH/g. According to the same manner as that of Example 1, this BMI was used to obtain a purified BMI (P-10) solution. The result of measurement of an acid value of P-10 is shown in Table 1.

### Example 11

As a crude BMI solution, a toluene solution (concentration: 50% by mass) of BMI-3000 which is commercially available from “DMI” was prepared. This BMI is BMI using DDA which was imide-extended with PMDA, as a diamine component, and had an acid value of 5.63 mg-KOH/g. According to the same manner as that of Example 1, this BMI was used to obtain a purified BMI (P-11) solution. The result of measurement of an acid value of P-11 is shown in Table 1.

### Example 12

According to the same manner as that of Example 1 except that a crude BMI solution was L-4, a purified BMI (P-12) solution was obtained. The result of measurement of an acid value of P-12 is shown in Table 1.

### Example 13

According to the same manner as that of Example I except that a crude BMI solution was L-5, a purified BMI (P-13) solution was obtained. The result of measurement of an acid value of P-13 is shown in Table 1.

### Example 14

According to the same manner as that of Example 1 except that in a crude BMI solution, 2 g of EDC was used as CDI, a purified BMI (P-14) solution was obtained. The result of measurement of an acid value of P-14 is shown in Table 1.

### Comparative Example 1

The same quantity of water and toluene were added to a crude BMI solution (L-1), and an operation of solvent extraction was performed two times, thereby, a purified BMI solution (R-1) was obtained by water washing. The result of measurement of an acid value of R-1 is shown in Table 1.

### Comparative Example 2

According to the same manner as that of Comparative Example 1 except that a crude BMI solution was L-2, a purified BMI solution (R-2) was obtained by water washing. The result of measurement of an acid value of R-2 is shown in Table 1.

### Comparative Example 3

The same quantity of 1% aqueous ammonia and toluene were added to a crude BMI solution (L-3), an operation of solvent extraction was performed two times, thereafter, solvent extraction with only water was performed two times, and thereafter, a purified BMI solution (R-3) was obtained by water washing. The result of measurement of an acid value of R-3 is shown in Table 1.

### Comparative Example 4

A liquid obtained by adding toluene to a crude BMI solution (L-1) was added to a large amount of methanol to reprecipitate BMI, the precipitate was collected, thereafter, it was mixed with toluene, thereby, a purified BMI (R-4) solution was obtained. The result of measurement of an acid value of R-4 is shown in Table 1.

### Comparative Example 5

A liquid obtained by adding toluene to a crude BMI solution (L-3) was added to a large amount of acetone to reprecipitate BMI, the precipitate was collected, thereafter, it was mixed with toluene, thereby, a purified BMI (R-5) solution was obtained. The result of measurement of an acid value of R-5 is shown in Table 1.

### Comparative Example 6

Each of crude BMI solutions (L-1) to (L-5) was cooled to −5° C. to precipitate BMI, this was tried to be purified by filtration, but BMI could not be precipitated from any solution.

### Comparative Example 7

According to the same manner as that of Comparative Example 1 except that an operation of solvent extraction was performed ten times, a purified BMI solution was obtained by water washing, but an acid value of this BMI remained almost unchanged from that of R-1.

### Comparative Example 8

In Comparative Example 4, a purification operation by reprecipitation was further performed five times, and a purified BMI solution was obtained, but an acid value of this BMI remained almost unchanged from that of R-4.

Results of measurement of an acid value of BMIs obtained in

Examples 1 to 14, and Comparative Examples 1 to 5 are shown in Table 1.

TABLE 1

| | | Acid value |
|---|---|---|
| | BMI | (mg-KOH/g) |
| Example 1 | P-1 | 0.80 |
| Example 2 | P-2 | 0.46 |
| Example 3 | P-3 | 1.35 |
| Example 4 | P-4 | 1.56 |
| Example 5 | P-5 | 0.71 |
| Example 6 | P-6 | 1.78 |
| Example 7 | P-7 | 0.91 |
| Example 8 | P-8 | 0.26 |
| Example 9 | P-9 | 0.39 |
| Example 10 | P-10 | 0.33 |
| Example 11 | P-11 | 0.51 |
| Example 12 | P-12 | 0.46 |
| Example 13 | P-13 | 0.37 |
| Example 14 | P-14 | 0.28 |
| Comparative Example 1 | R-1 | 8.85 |
| Comparative Example 2 | R-2 | 8.21 |
| Comparative Example 3 | R-3 | 8.56 |
| Comparative Example 4 | R-4 | 7.33 |
| Comparative Example 5 | R-5 | 7.21 |

As shown in Examples and Comparative Examples, it is seen that BMI of the present invention has been remarkably reduced in an acid value thereof, as compared with BMI which has been purified by the known purification method, and BMI of the present invention has an acid value of 2 mg-KOH/g or less. INDUSTRIAL APPLICABILITY

Since BMI of the present invention has a remarkably low acid value, it is excellent in a corrosion resistance, and has a low hygroscopicity. Accordingly, it is useful as a laminate material, a seal material, an electrical insulating material, an electrically conductive paste, an adhesive, a pressure-sensitive adhesive, a structural material and the like.

1. A bismaleimide using an aliphatic diamine as a diamine component, characterized in that an acid value thereof is 2 mg-KOH/g or less.

2. A method for producing the bismaleimide as defined in claim 1, comprising:
adding a carbodiimide to a solution comprising a crude bismaleimide having an acid value of more than 2 mg-KOH/g; and
reacting acid components in the crude bismaleimide with the carbodiimide to give a bismaleimide having an acid value of 2 mg-KOH/g or less.

3. A method for producing the bismaleimide as defined in claim 1, comprising:
adding a carbodiimide to a solution comprising a crude bismaleimide having an acid value of more than 2 mg-KOH/g;
reacting acid components in the crude bismaleimide with the carbodiimide; and
thereafter, removing a urea derivative of the carbodiimide.

4. A method for producing the bismaleimide of claim 3, wherein the urea derivative of the carbodiimide is soluble in methanol.

5. A method for producing the bismaleimide of claim 3, wherein the method of removing the urea derivative of the carbodiimide is a solvent extraction method.
